(19) United States
(12) Patent Application Publication
(10) Pub. No.: US 2011/0105794 A1
(43) Pub. Date: May 5, 2011

(54) Process for the Isomerization of Semicarbazone Compounds

(75) Inventors: Thomas Zierke, Boehl-Iggelheim (DE); Stefan Engel, Nieder-Olm (DE)

(73) Assignee: BASF SE, Ludwigshafen (DE)

(21) Appl. No.: 13/001,063

(22) PCT Filed: Jun. 22, 2009
(86) PCT No.: PCT/EP09/57727

§ 371 Date: Dec. 23, 2010

(30) Foreign Application Priority Data
Jun. 30, 2008 (EP) 08159347.7

(51) Int. Cl. C07C 237/18

(52) U.S. Cl. 564/168

(57) ABSTRACT

The present invention relates to a process for the isomerization of the Z-isomer I-Z of a semicarbazone compound of the general formula (I) into its E-isomer I-E

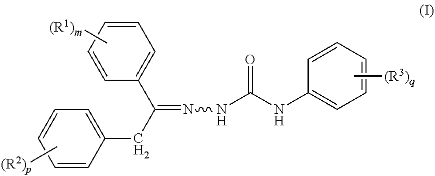

wherein the variables in formula (I) have the meanings as defined in the description.

The present invention relates to a process for the isomerization of the Z-isomer I-Z of semicarbazone compounds of the general formula I into its E-isomer I-E

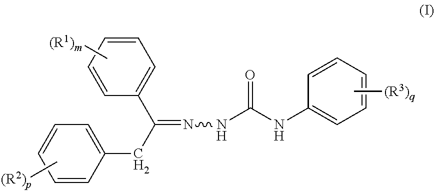

wherein the variables in formula I have the following meanings:
m, p and q are each independently an integer of 0, 1, 2, 3 or 4;
R¹, R², R³ are each independently halogen; OH; CN; NO₂;
   C₁-C₆-alkyl, optionally substituted with C₁-C₄-alkoxy, C₁-C₄-haloalkoxy or C₃-C₆-cycloalkyl;
   C₁-C₆-haloalkyl;
   C₃-C₆-cycloalkyl;
   C₁-C₆-alkoxy, optionally substituted with C₁-C₄-alkoxy or
   C₃-C₆-cycloalkyl;
   C₁-C₆-haloalkoxy;
   C₁-C₆-alkylcarbonyl;
   C₃-C₆-cycloalkoxy;
   C₁-C₆-alkoxycarbonyl or
   C₁-C₆-alkoxycarbonyloxy.

Semicarbazone compounds of the general formula I are known from EP-A-462456 to be effective as pest-controlling agents. Semicarbazones of the formula I have two geometrical isomers with regard to the C=N-double bond, namely the E-form I-E and the Z-form I-Z.

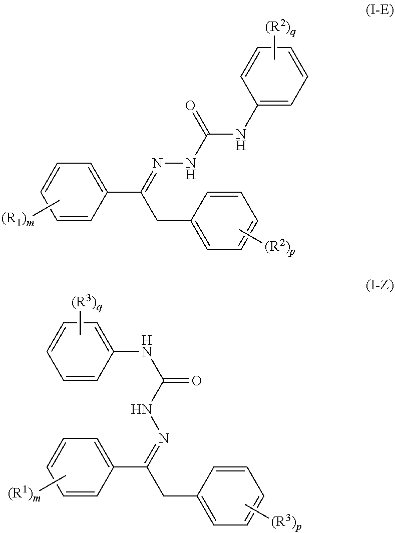

At room temperature these geometrical isomers are stable with regard to E/Z-isomerization. As regards the relative pesticidal activity of these compounds, the E-form I-E is generally more active than the Z-form I-Z. Therefore, agriculturally and commercially acceptable specifications of semicarbazones I require an E/Z-ratio of at least 9:1 and preferably at least 10:1.

Compounds of the formula I can be prepared by the process illustrated in the following scheme:

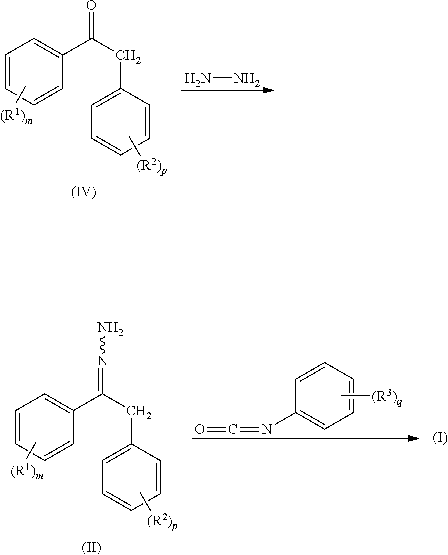

Significant amounts of the undesired Z-isomer I-Z are formed by this process. Moreover, much effort is needed to achieve the desired E/Z-ratio. Firstly, long reaction times are required to achieve a high E/Z-ratio in the hydrazone precursor II, necessary for obtaining the desired E/Z-ratio in the final product I. Secondly, the crystallization of the E-isomer I-E in the presence of the Z-isomer I-Z is tedious and difficult. In order to obtain a high isolated yield of the desired E-isomer, some of the Z-isomer must also be crystallized with the E-isomer from the reaction mixture. Similarly, in order to obtain the desired E/Z-ratio in the crystallized product, a low isolated yield of the E-isomer is necessary, so that the undesired Z-isomer is completely solubilized along with significant amount of E-isomer in the reaction mixture. Thirdly, recrystallization of isolated product I containing significant amounts of the undesired Z-isomer to obtain the desired E/Z ratio is also tedious and difficult. As with crystallization from the reaction mixture, either low crystallization recoveries or high Z-isomer content of the final product are obtained. These involve the risk of either isolating a product in low yield or not having the required E/Z-ratio.

WO 2005/047235 A1 discloses a process for the isomerization of the semicarbazones I to the favored E-isomer in the presence of iodine which is advantageously performed in the solid or molten phase. A disadvantage of this process is that a large-scale production of the E-enriched semicarbazones I would require special and expensive process equipment suited to heat up solid material to the desired temperature. Moreover, the use of iodine is not attractive from a manufacturing point of view due to toxicological and corrosion problems. For example, iodine waste must be treated as hazardous waste.

Therefore, there remains a need to provide a process for the isomerization of the Z-isomer of I into its E-isomer I-E, which is more convenient to practice and more feasible from an environmental and economical point of view.

Surprisingly, it has been found that the Z-isomer of the compound I can be isomerized into the E-isomer of I by reacting the Z-form I-Z or a mixture of the geometric isomers I-E and I-Z in the presence of at least one organic acid. This constitutes a quite astonishing result given the fact that, in the preparation of the hydrazone precursor II, acceptable E/Z ratios with a view to the pesticidal use of the final product I could not be achieved in the presence of organic acids.

Therefore, the present invention relates to a process for the isomerization of the Z-isomer I-Z of a compound of the general formula I as defined above into its E-isomer I-E by reacting the Z-isomer I-Z or a mixture of the geometrical isomers I-Z and I-E in the presence of at least one organic acid.

The organic acid used in the process of this invention can in principle be any organic acid.

In a preferred embodiment, the organic acid is selected from carboxylic acids and sulfonic acids.

As used herein, the term “carboxylic acids” refers to, e.g., aliphatic carboxylic acids and aromatic carboxylic acids, each of which may be unsubstituted or substituted.

As used herein, the term “sulfonic acids” refers to, e.g., aliphatic sulfonic acids and aromatic sulfonic acids, each of which may be unsubstituted or substituted.

Preferably, the organic acid is selected from aliphatic carboxylic acids, aromatic carboxylic acids, aliphatic sulfonic acids, aromatic sulfonic acids and any mixtures thereof, in each case being unsubstituted or substituted.

The aliphatic carboxylic acid is preferably selected from alkyl carboxylic acids wherein the alkyl group is C₁-C₄-alkyl being unsubstituted or substituted with one or more halogen atoms. More preferably, the aliphatic carboxylic acid is selected from alkyl carboxylic acids wherein the alkyl group is C₁-C₄-alkyl being substituted with one or more halogen atoms independently selected from fluorine, chlorine or bromine (more preferably from chlorine or fluorine). It is particularly preferred that the aliphatic carboxylic acid is selected from alkyl carboxylic acids wherein the alkyl group is C₁-C₂-alkyl substituted with 1 to 5 fluorine atoms (also referred to herein as “C₁-C₂-fluoroalkyl”). Examples of the aforementioned aliphatic carboxylic acids are formic acid, acetic acid, chloroacetic acid, chlorodifluoroacetic acid, dichloroacetic acid, difluoroacetic acid, trichloroacetic acid, trifluoroacetic acid and any mixtures thereof, with trifluoroacetic acid being preferred.

The aromatic carboxylic acid is preferably selected from aryl carboxylic acids wherein the aryl group is unsubstituted or substituted with one or more substituents independently selected from C₁-C₆-alkyl, C₁-C₆-haloalkyl, halogen or nitro. More preferably, the aromatic carboxylic acid is selected from aryl carboxylic acids wherein the aryl group is unsubstituted or substituted with one or more substituents independently selected from C₁-C₆-haloalkyl or halogen. In an even more preferred embodiment, the aromatic carboxylic acid is selected from aryl carboxylic acids wherein the aryl group is phenyl being unsubstituted or substituted with one to three substituents independently selected from C₁-C₄-haloalkyl or halogen. In yet another preferred embodiment, the aromatic carboxylic acid is selected from aryl carboxylic acids wherein the aryl group is phenyl being unsubstituted or substituted with one to three substituents independently selected from C₁-C₂-fluoroalkyl or chlorine. In an even more preferred embodiment, the aromatic carboxylic acid is selected from aryl carboxylic acids wherein the aryl group is phenyl being unsubstituted or substituted with one or two substituents independently selected from C₁-C₂-fluoroalkyl (in particular trifluoromethyl) or chlorine. Examples of the aforementioned aromatic carboxylic acids are benzoic acid, o-methylbenzoic acid, m-methylbenzoic acid, p-methylbenzoic acid, p-tert-butylbenzoic acid, o-trifluoromethyl benzoic acid, m-trifluoromethyl benzoic acid, p-trifluoromethyl benzoic acid, o-chlorobenzoic acid, m-chlorobenzoic acid, p-chlorobenzoic acid, o-nitrobenzoic acid, m-nitrobenzoic acid, p-nitrobenzoic acid and any mixtures thereof. Preferred aromatic carboxylic acids include benzoic acid, o-trifluoromethyl benzoic acid, m-trifluoromethyl benzoic acid, p-trifluoromethyl benzoic acid, o-chlorobenzoic acid, m-chlorobenzoic acid, p-chlorobenzoic acid and any mixtures thereof. More preferably, the aromatic carboxylic acid is selected from benzoic acid, m-trifluoromethyl benzoic acid, o-chlorobenzoic acid, m-chlorobenzoic acid, p-chlorobenzoic acid and any mixtures thereof.

The term “aryl” as used herein refers to an aromatic carbocyclic group having at least one aromatic ring (e.g., phenyl or biphenyl) or multiple condensed rings in which at least one ring is aromatic (e.g., 1,2,3,4-tetrahydronaphthyl, naphthyl, anthryl, or phenanthryl), each of which may be substituted.

Preferred aliphatic sulfonic acids are alkyl sulfonic acids wherein the alkyl group is C₁-C₄-alkyl being unsubstituted or substituted with one or more halogen atoms, in particular fluorine. More preferably, the aliphatic sulfonic acid is selected from alkyl sulfonic acids wherein the alkyl group is C₁-C₂-alkyl which is unsubstituted or substituted with 1 to 5 fluorine atoms. Suitable aliphatic sulfonic acids are, for example, methanesulfonic acid, ethanesulfonic acid and trifluoromethanesulfonic acid, with methanesulfonic acid being preferred.

Preferably, the aromatic sulfonic acid is selected from aryl sulfonic acids wherein the aryl group is unsubstituted or substituted with one or more substituents independently selected from C₁-C₆-alkyl or halogen. More preferably, the aromatic sulfonic acid is selected from aryl sulfonic acids wherein the aryl group is phenyl being unsubstituted or substituted with one or more substituents independently selected from C₁-C₆-alkyl or halogen. It is even more preferred that the aromatic sulfonic acid is selected from aryl sulfonic acids wherein the aryl group is phenyl being unsubstituted or substituted with one to three C₁-C₆-alkyl, preferably one or two C₁-C₄-alkyl. Examples of the aforementioned aromatic sulfonic acids are benzenesulfonic acid, o-toluenesulfonic acid, m-toluene sulfonic acid, p-toluenesulfonic acid, 2,5-dimethylbenzenesulfonic acid, 3,4-dimethylbenzenesulfonic acid, m-xylenesulfonic acid, o-ethylbenzene sulfonic acid, m-ethylbenzene sulfonic acid, p-ethylbenzene sulfonic acid, 4-chlorobenzenesulfonic acid and any mixtures thereof. Preferred aromatic sulfonic acids are benzenesulfonic acid and p-toluenesulfonic acid, with p-toluenesulfonic acid being most preferred.

In a preferred embodiment, the organic acid is selected from alkyl carboxylic acids wherein the alkyl group is C₁-C₄-alkyl being unsubstituted or substituted with one or more halogen atoms, aryl carboxylic acids wherein the aryl group is unsubstituted or substituted with one or more substituents independently selected from C₁-C₆-alkyl, C₁-C₆-haloalkyl, halogen or nitro, alkyl sulfonic acids wherein the alkyl group is C₁-C₄-alkyl being unsubstituted or substituted with one or more halogen atoms and aryl sulfonic acids wherein the aryl group is unsubstituted or substituted with one or more substituents independently selected from C₁-C₆-alkyl or halogen.

In another preferred embodiment, the organic acid is selected from formic acid, acetic acid, chloroacetic acid, chlorodifluoroacetic acid, dichloroacetic acid, difluoroacetic acid, trichloroacetic acid, trifluoroacetic acid, benzoic acid, o-methylbenzoic acid, m-methylbenzoic acid, p-methylbenzoic acid, p-tert-butylbenzoic acid, o-trifluoromethyl benzoic acid, m-trifluoromethyl benzoic acid, p-trifluoromethyl benzoic acid, o-chlorobenzoic acid, m-chlorobenzoic acid, p-chlorobenzoic acid, o-nitrobenzoic acid, m-nitrobenzoic acid, p-nitrobenzoic acid, methanesulfonic acid, ethanesulfonic acid, trifluoromethanesulfonic acid, benzenesulfonic acid, o-toluenesulfonic acid, m-toluene sulfonic acid, p-toluenesulfonic acid, 2,5-dimethylbenzenesulfonic acid, 3,4-dimethylbenzenesulfonic acid, m-xylenesulfonic acid, o-ethylbenzene sulfonic acid, m-ethylbenzene sulfonic acid, p-ethylbenzene sulfonic acid, 4-chlorobenzenesulfonic acid and any mixtures thereof.

In an even more preferred embodiment, the organic acid is selected from trifluoroacetic acid, benzoic acid, m-trifluoromethyl benzoic acid, o-chlorobenzoic acid, m-chlorobenzoic acid, p-chlorobenzoic acid, methanesulfonic acid, benzenesulfonic acid, p-toluenesulfonic acid and any mixtures thereof.

In general, at least 0.05% by weight, preferably at least 0.1% by weight or more preferably at least 0.2% by weight of the at least one organic acid, based on the total weight of the compound I, are required to achieve an isomerization. For practical reasons, the amount of the organic acid will generally not exceed 5% by weight, especially not 2% by weight and in particular not 1% by weight, based on the total weight of the compound I. In a preferred embodiment, the organic acid is used in an amount of from 0.1 to 5% by weight, based on the total weight of the compound I. More preferably, the organic acid is used in an amount of from 0.1 to 2% by weight, based on the total weight of the compound I. It is even more preferred when the process of this invention is carried out in the presence of 0.1 to 1% by weight of the organic acid, based on the total weight of the compound I.

The temperature at which the process of this invention is carried out will be at least 30° C., preferably at least 40° C. and more preferably at least 45° C. The process of this invention is preferably effected at temperatures below 110° C., especially below 90° C. and most preferably below 80° C. It is especially preferred when the isomerization is performed at temperatures in the range of 40° C. to 90° C., especially in the range of 45° C. to 80° C. and in particular in the range of 45° C. to 70° C.

Conveniently, in the isomerization the concentration and temperature are chosen such that the E-isomer I-E formed is separated continuously from the reaction medium.

The process of the invention can be performed by using almost pure Z-isomer (E/Z-ratio <5:95) or mixtures of the geometrical isomers I-E and I-Z (E/Z-ratio >5:95) as the starting material. In a preferred embodiment of the present invention, a mixture of the geometrical isomers I-E and I-Z having an E/Z-ratio ranging from 1:1 to 15:1, preferably 2:1 to 15:1 and especially from 3:1 to 10:1 is used as the starting material.

In general, the isomerization of the compound I is performed until a E/Z ratio of at least 30:1, preferably at least 50:1 and more preferably at least 80:1 is obtained. The reaction time, which is required to achieve the desired E/Z-ratio varies with the amount and type of organic acid, which is used, and is in the range of 1 to 20 h, preferably 1 to 15 h and more preferably 2 to 10 h.

The isomerization may be performed in an inert organic solvent or diluent. Suitable organic solvents are aromatic solvents such as benzene, toluene, xylenes (i.e. m-xylene, o-xylene, p-xylene, and any mixture thereof), chlorobenzene and dichlorobenzene; acyclic ethers such as diethyl ether and methyl-tert.-butyl ether; alicyclic ethers such as tetrahydrofurane and dioxane; alkanols such as methanol, ethanol, propanol, isopropanol and n-butanol; ketones such as acetone and methylethyl ketone; nitriles such as acetonitrile and propionitrile; carbonates such as dimethylcarbonate, diethylcarbonate, ethylene carbonate and propylene carbonate; aliphatic and alicyclic hydrocarbons such as hexane, isohexane, heptane and cyclohexane; and mixtures of the aforementioned solvents. Preferred solvents are the aforementioned aromatic solvents, especially alkylbenzenes and even more preferably alkylbenzenes which are mono-, di-, or trialkylsubstituted with each alkyl group containing 1 to 3 carbon atoms, in particular toluene, xylenes and mixtures of the aforementioned solvents which contain at least 50% by volume of the aforementioned aromatic solvents.

In order to perform the isomerization in an inert organic solvent or diluent, the Z-isomer I-Z or a mixture of the geometrical isomers I-E and I-Z can be dissolved or suspended in a suitable solvent or mixtures of solvents and reacted in the presence of the at least one organic acid as outlined above. It is particularly advantageous that the isomerization is carried out in a suspension of the Z-isomer I-Z or of a mixture of the geometrical isomers I-E and I-Z in any of the aforementioned organic solvents or any mixture thereof. Said suspension may already contain the organic acid. Preferably, the organic acid is added to the suspension as this enables a fast and efficient isomerization.

It is also possible to perform the isomerization either in the reaction mixture obtained from the reaction of the hydrazone II and the isocyanate III or in the mother liquor obtained after crystallization of the compound I from the reaction mixture.

In order to obtain the E-isomer I-E, optionally together with small amounts of Z-isomer I-Z, the isomerization mixture is worked-up in a usual manner. Preferably, the isomer I-E, optionally together with small amounts of isomer I-Z (in general not more than 5% by weight) is isolated from the liquid reaction mixture by crystallization or precipitation. Crystallization or precipitation may be achieved either by cooling and/or concentration of the liquid reaction mixture and/or by the addition of an inert solvent which decreases the solubility of the compound I in the reaction mixture. Suitable solvents for decreasing the solubility of the compound I are aliphatic or alicyclic hydrocarbons such as hexane, heptane, isohexane and cyclohexane.

The isomerization of I-Z may also be performed in the absence of a solvent or diluent. In other words, the isomerization of the Z-isomer I-Z is performed in the solid phase or in the melt-phase. Thus, the solid or molten compound I-Z or a solid or molten mixture of the geometrical isomers I-E and I-Z is reacted with the at least one organic acid as outlined above. After the desired degree of isomerization is achieved, the at least one organic acid can be simply removed by sublimation, e.g. by increasing the temperature and/or by applying reduced pressure. The residue usually contains only compound I having an increased E/Z-ratio with regard to the starting material and optionally those impurities contained in the starting material. The residue usually does not contain any further impurities.

Starting materials for the isomerization in the absence of a solvent or diluent may be the pure Z-isomer or mixtures of the geometrical isomers I-E and I-Z. Examples of such mixtures are crystalline products which do not fulfil the required E/Z-ratio and the residue obtained from the mother liquor of the crystallization of compound I during the work-up in the preparation of compound I.

The organic moieties mentioned in the above definitions of the variables are—like the term halogen—collective terms for individual listings of the individual group members. The prefix Cₙ-Cₘ indicates in each case the possible number of carbon atoms in the group. The term halogen denotes in each case fluorine, bromine, chlorine or iodine, preferably fluorine, bromine or chlorine and in particular fluorine or chlorine. Examples of other meanings are:

The term “C₁-C₆-alkyl” as used herein and the alkyl moieties of C₁-C₆-alkoxy, C₁-C₆-alkoxycarbonyl, C₁-C₆-alkylcarbonyl, and C₁-C₆-alkoxycarbonyloxy refer to a saturated straight-chain or branched hydrocarbon group having from 1 to 6 carbon atoms, especially from 1 to 4 carbon groups, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. C₁-C₄-alkyl means for example methyl, ethyl, propyl, 1-methylethyl, butyl, 1-methylpropyl, 2-methylpropyl or 1,1-dimethylethyl.

In C₁-C₆-alkyl one hydrogen may be substituted by a radical, selected from C₁-C₄-alkoxy, C₁-C₄-haloalkoxy and C₃-C₆-cycloalkyl.

The term “C₁-C₆-haloalkyl” as used herein refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “C₁-C₂-fluoroalkyl” as used herein refers to a C₁-C₂-alkyl which carries 1, 2, 3, 4 or 5 fluorine atoms, for example difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “C₁-C₆-alkoxy” as used herein and the alkoxy moieties of C₁-C₆-alkoxycarbonyl, and C₁-C₆-alkoxycarbonyloxy refers to a straight-chain or branched saturated alkyl group having 1 to 6 carbon atoms (as mentioned above) which is attached via an oxygen atom to the remainder of the molecule. Examples include methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

In C₁-C₆-alkoxy one hydrogen may be substituted by a radical, selected from C₁-C₆-alkoxy and C₃-C₆-cycloalkyl.

The term “C₁-C₆-haloalkoxy” as used herein refers to a C₁-C₆-alkoxy group as mentioned above which is partially or fully substituted by fluorine, chlorine, bromine and/or iodine, i.e., for example, C₁-C₆-haloalkoxy such as chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy, in particular chloromethoxy, fluoromethoxy, difluoromethoxy, trifluoromethoxy, 2-fluoroethoxy, 2-chloroethoxy or 2,2,2-trifluoroethoxy.

The term “C₃-C₆-cycloalkyl” as used herein refers to a cycloaliphatic radical having from 3 to 6 carbon atoms, such as cyclopropyl, cyclobutyl, cyclopentyl and cyclohexyl. The cycloalkyl radical may be unsubstituted or may carry 1 to 6 C₁-C₄ alkyl radicals, preferably a methyl radical.

In general, the isomerization can be performed on any of the compounds of the formula I. In a preferred embodiment of the invention the variables m, p and q are each 1.

Preferred radicals R¹, R², R³ are each independently halogen, CN, C₁-C₆-alkyl, C₁-C₆-haloalkyl, C₁-C₆-alkoxy or C₁-C₆-haloalkoxy. More preferably R¹ is halogen or C₁-C₄-haloalkyl, especially CF₃, R² is CN and R³ is C₁-C₄-haloalkoxy, especially OCF₃.

An example of an especially preferred compound I is a compound where R¹ is CF₃ located in the 3-position of the phenyl ring, R² is CN located in the 4-position of the phenyl ring and R³ is OCF₃ located in the 4-position of the phenyl ring. This compound is referred to as I.1, the isomers are referred to as I.1-E and I.1-Z:

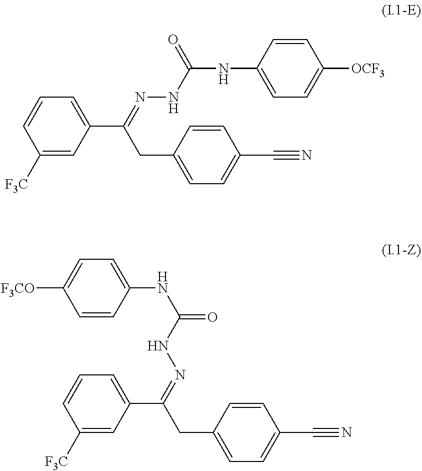

The process of the present invention allows an easy isomerization of the Z-isomer I-Z into its E-isomer I-E. The isomerization usually yields a high E/Z-ratio which exceeds 95:5, preferably 97:3 and more preferably 98:2. No noticeable amounts of byproducts are formed, i.e. the yield of compound I is >99%. Therefore, the process of the present invention can be used to simplify the preparation of compounds I with the desired E/Z-ratio of Z 9:1 and to enhance the overall isolated yield which is particularly beneficial from an economical point of view.

Combinations of specific or preferred embodiments with other specific or preferred embodiments are within the scope of the present invention.

The following examples are intended to illustrate the present invention without limiting its scope.

### EXAMPLE 1

### Treatment of Crude Compound I.1 Having an E/Z Ratio of 4.8:1 with p-Toluene Sulfonic Acid in Toluene

2 g of a solid containing 90 wt-% of compound I.1 having an E/Z ratio of 4.8:1 (according to HPLC evaluation, by area-%) were suspended in 3.5 g of toluene together with 0.1 g of p-toluene sulfonic acid and the resulting slurry was heated to 50° C. for 4 h. Then the reaction mixture was cooled and filtered. The filter cake was washed with 10 g of cyclohexane. 18 g of a wet solid were thus obtained. The E/Z ratio was determined by HPLC to be 204:1 (area-% evaluation).

### EXAMPLE 2

### Treatment of Crude Compound I.1 Having a E/Z Ratio of 4.8:1 with p-Toluene Sulfonic Acid in o-Xylene

2 g of a solid containing 90 wt-% of compound I.1 having an E/Z ratio of 4.8:1 (according to HPLC, area-% evaluation) were suspended in 3.5 g of o-xylene together with 0.1 g of p-toluene sulfonic acid and the resulting slurry was heated to 50° C. for 4 h. Then the reaction mixture was cooled and filtered. The filter cake was washed with 10 g of cyclohexane. 1.8 g of a wet solid were thus obtained. The E/Z ratio was determined by HPLC to be 54:1 (area-% evaluation).

### EXAMPLE 3

### Treatment of Crude Compound I.1 Having a E/Z Ratio of 4.8:1 with Benzoic Acid in Toluene

2 g of a solid containing 90 wt-% of compound I.1 having an E/Z ratio of 4.8:1 (according to HPLC, area-% evaluation) were suspended in 3.5 g of toluene together with 0.1 g of benzoic acid and the resulting slurry was heated to 50° C. for 4 h. Then the reaction mixture was cooled and filtrated. The filter cake was washed with 10 g of cyclohexane. 1.8 g of a wet solid were thus obtained. The E/Z ratio was determined by HPLC to be 103:1 (area-% evaluation).

### EXAMPLE 4

### Treatment of Crude Compound I.1 Having a E/Z Ratio of 4.8:1 with Benzoic Acid in o-Xylene

2 g of a solid containing 90 wt-% of compound I.1 having an E/Z ratio of 4.8:1 (according to HPLC, area-% evaluation) were suspended in 3.5 g of o-xylene together with 0.1 g of benzoic acid and the resulting slurry was heated to 50° C. for 4 h. Then the reaction mixture was cooled and filtered. The filter cake was washed with 10 g of cyclohexane. Thus 1.9 g of a wet solid were obtained. The E/Z ratio was determined by HPLC to be 24:1 (area-% evaluation).

### EXAMPLE 5

### Treatment of a Reaction Mixture of Compound I.1 Obtained from the Corresponding Hydrazone II.1 (i.e. Hydrazone II Wherein R¹ is CF₃ Located in the 3-Position of the Phenyl Ring and R² is CN Located in the 4-Position of the Phenyl Ring) and the Isocyanate III.1 (i.e. Isocyanate III Wherein R³ is OCF₃ Located in the 4-Position of the Phenyl Ring) with p-Toluene Sulfonic Acid

After reacting 217 mmol of the hydrazone II.1 with excess isocyanate III.1 at 90° C. in 180 g of toluene and destroying the excess isocyanate III.1 by addition of methanol, the reaction mixture was cooled to 50° C. During cooling product precipitation was observed. A sample for HPLC analysis (wt-% evaluation) was taken from the reaction mixture immediately before the addition of p-toluene sulfonic acid. 243 mg of p-toluene sulfonic acid were then added to the reaction mixture and the mixture was held at 50° C. for 18.5 h. Samples for HPLC analysis (wt-% evaluation) were taken from this mixture at different time intervals after addition of p-toluene sulfonic acid. The results are presented in the following table:

| Time* (h) | Amount I-E (wt-%) | Amount I-E (wt-%) | E/Z-ratio |
|---|---|---|---|
| 0 | 3.5 | 30.2 | 8.0:1 |
| 1.0 | 1.14 | 34.1 | 30.1:1 |
| 2.5 | 0.43 | 33.6 | 77.8:1 |
| 3.5 | 0.39 | 33.8 | 87.7:1 |
| 5.0 | 0.39 | 33.6 | 87.2:1 |

*after addition of p-toluene sulfonic acid

The mixture was cooled down to 10° C. and held at this temperature for 6 h. The product was filtered, washed with 87 g toluene and dried under vacuum at 80° C. Yield: 101 g Compound I.1 (I-E: 97.5 wt-%; I-Z: 0.6 wt-%), i.e. 90% of theory.

In the above examples 1 to 5, the high-performance liquid chromatography (HPLC) evaluations were performed using the following conditions:

column: Machery Nagel, CC 150/4,6 Kromasil, 100-3,5 C8; oven temperature: 35° C.,
eluent: Acetonitril/water (buffered to pH 2.4; 0.05% trifluoroacetic acid/ammonia) gradient; flow rate: 0.4 ml/min, detection: UV 235 nm

1-12. (canceled)

13: A process for the isomerization of the Z-isomer I-Z of a compound of the general formula I into its E-isomer I-E 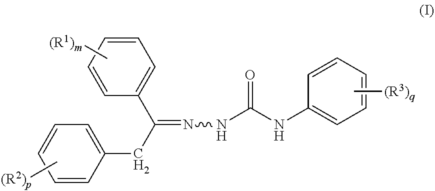
wherein
m, p and q are each independently an integer of 0, 1, 2, 3 or 4
R¹, R², R³ are each independently halogen; OH; CN; NO₂;
C₁-C₆-alkyl, optionally substituted with C₁-C₄-alkoxy, C₁-C₄-haloalkoxy or C₃-C₆-cycloalkyl;
C₁-C₆-haloalkyl;
C₃-C₆-cycloalkyl;
C₁-C₆-alkoxy optionally substituted with C₁-C₄-alkoxy or
C₃-C₆-cycloalkyl;
C₁-C₆-haloalkoxy;
C₁-C₆-alkylcarbonyl;
C₃-C₆-cycloalkoxy;
C₁-C₆-alkoxycarbonyl or
C₁-C₆-alkoxycarbonyloxy;
said process comprising reacting the Z-isomer I-Z or a mixture of the stereoisomers I-Z and I-E in the presence of at least one organic acid.

14: The process of claim 13, wherein the organic acid is selected from the group consisting of carboxylic acids and sulfonic acids.

15: The process of claim 13, wherein the organic acid is selected from the group consisting of aliphatic carboxylic acids, aromatic carboxylic acids, aliphatic sulfonic acids, aromatic sulfonic acids and any mixtures thereof, in each case being unsubstituted or substituted.

16: The process of claim 13, wherein the organic acid is selected from the group consisting of alkyl carboxylic acids wherein the alkyl group is C₁-C₄-alkyl being unsubstituted or substituted with one or more halogen atoms, aryl carboxylic acids wherein the aryl group is unsubstituted or substituted with one or more substituents independently selected from C₁-C₆-alkyl, C₁-C₆-haloalkyl, halogen or nitro, alkyl sulfonic acids wherein the alkyl group is C₁-C₄-alkyl being unsubstituted or substituted with one or more halogen atoms and aryl sulfonic acids wherein the aryl group is unsubstituted or substituted with one or more substituents independently selected from C₁-C₆-alkyl or halogen.

17: The process of claim 13, wherein the organic acid is selected from the group consisting of formic acid, acetic acid, chloroacetic acid, chlorodifluoroacetic acid, dichloroacetic acid, difluoroacetic acid, trichloroacetic acid, trifluoroacetic acid, benzoic acid, o-methylbenzoic acid, m-methylbenzoic acid, p-methylbenzoic acid, p-tert-butylbenzoic acid, o-trifluoromethyl benzoic acid, m-trifluoromethyl benzoic acid, p-trifluoromethyl benzoic acid, o-chlorobenzoic acid, m-chlorobenzoic acid, p-chlorobenzoic acid, o-nitrobenzoic acid, m-nitrobenzoic acid, p-nitrobenzoic acid, methanesulfonic acid, ethanesulfonic acid, trifluoromethanesulfonic acid, benzenesulfonic acid, o-toluenesulfonic acid, m-toluene sulfonic acid, p-toluenesulfonic acid, 2,5-dimethylbenzenesulfonic acid, 3,4-dimethylbenzenesulfonic acid, m-xylenesulfonic acid, o-ethylbenzene sulfonic acid, m-ethylbenzene sulfonic acid, p-ethylbenzene sulfonic acid, 4-chlorobenzenesulfonic acid and any mixtures thereof.

18: The process of claim 13, wherein the organic acid is selected from the group consisting of trifluoroacetic acid, benzoic acid, m-trifluoromethyl benzoic acid, o-chlorobenzoic acid, m-chlorobenzoic acid, p-chlorobenzoic acid, methanesulfonic acid, benzenesulfonic acid, p-toluenesulfonic acid and any mixtures thereof.

19: The process of claim 13, wherein the organic acid is used in amounts from 0.1 to 5% by weight, based on the total weight of the compound of the general formula I.

20: The process of claim 13, wherein the isomerization is performed in an inert organic solvent or diluent.

21: The process of claim 13, wherein a mixture of the isomers I-Z and I-E having an E/Z ratio ranging from 15:1 to 1:1 is reacted.

22: The process of claim 13, wherein the isomerization is performed at a temperature ranging from 40° C. to 90° C.

23: The process of claim 13, wherein in formula I
m, p and q are each 1 and
R¹, R², R³ are each independently halogen, CN, C₁-C₆-alkyl, C₁-C₆-haloalkyl, C₁-C₆-alkoxy or C₁-C₆-haloalkoxy.

24: The process of claim 23, wherein in formula I
R¹ is CF₃ located at the 3-position of the phenyl ring,
R² is CN located at the 4-position of the phenyl ring and
R³ is OCF₃ located at the 4-position of the phenyl ring.
